(19) United States
(12) Patent Application Publication
(10) Pub. No.: US 2004/0167340 A1
(43) Pub. Date: Aug. 26, 2004

(54) Process for preparing storage-stable benzothiazolyl sulfenamides

(76) Inventors: Stefan Grabowski, Dormagen (DE); Leon Felix, Temse (BE); Domien Sluyts, Hoevenen (BE); Patrick Missiaen, Edegem (BE); Reinhard Preuss, Brasschaat (BE)

Correspondence Address: BAYER POLYMERS LLC, 100 BAYER ROAD, PITTSBURGH, PA, 15205, US

(21) Appl. No.: 10/778,437

(22) Filed: Feb. 13, 2004

(30) Foreign Application Priority Data
Feb. 20, 2003 (DE) 10307138.5

(51) Int. Cl. C07D 277/68

(52) U.S. Cl. 548/166

(57) ABSTRACT

The present invention provides a process for preparing benzothiazolyl sulfenamides by reacting primary amines with alkali metal salts of mercaptobenzthiazole in the presence of hydrogen peroxide and alkali metal hypochlorite. The process according to the present invention is characterized by high rates of conversion, wherein the benzothiazolyl sulfenamides obtained are obtained with high purity, in high yields and with high storage-stability, even at elevated temperatures and high humidity.

### FIELD OF THE INVENTION

The present invention provides a process for preparing benzothiazolyl sulfenamides by reacting primary amines with alkali metal salts of mercaptobenzthiazole in the presence of hydrogen peroxide and alkali metal hypochlorite. The process according to the present invention is characterized high rates of conversion, wherein the benzothiazolyl sulfenamides obtained are obtained with high purity, in high yields and with high storage-stability, even at elevated temperatures and high humidity.

### BACKGROUND OF THE INVENTION

It is known that benzothiazolyl sulfenamides, which are used in particular as vulcanization accelerators in the rubber processing industry, can be prepared by the mutual oxidation of mercaptobenzthiazoles or their alkali metal salts and amines. Chlorine, hypochlorite or hydrogen peroxide, for example, are used as oxidizing agents.

Thus, for example, a process for preparing benzothiazolyl sulfenamides is described in European patent application 0 180 869-A2 wherein these are obtained by the oxidation in aqueous medium of mercaptobenzthiazole or its salts and primary amines.

The disadvantage of the process described in the European patent application mentioned, however, is that when oxidizing with hydrogen peroxide only up to 75% of the mercaptobenzthiazole used can be converted, in order to obtain the purity and yields described in the examples in the patent application. However, since the mercaptobenzthiazole can be converted at only a relatively low rate of conversion, this decreases the economic viability of the process described in the European patent application.

The disadvantage of the oxidation process using hypochlorite described is the large amount of chloride ions which are formed during the process and which, accordingly, has to be disposed of in an appropriate manner. In addition, this process is associated with many problems due to corrosion.

U.S. Pat. No. 5,436,346 also describes a process for preparing benzothiazolyl sulfenamides, by reacting 2-mercaptobenzthiazole with primary or secondary or cycloaliphatic amines in aqueous medium in the presence of hydrogen peroxide as an oxidizing agent.

The disadvantage of this process is that 2-mercaptobenzthiazoles are used as the starting products. These have to be prepared first from the corresponding alkali metal salts, this process naturally being accompanied by corresponding losses in yield due to the purification process. In addition, the performance of the process described in the U.S. patent on an industrial scale is complicated and therefore not very economic.

Thus, the object of the present invention is to provide an ecologically and economically improved process, starting from the alkali metal salts of mercaptobenzthiazoles, which leads to storage-stable products (at elevated temperatures and high humidity) and which also enables the use of starting products which are available in technical grade quality (about 93 to 97% strength). In addition, the present invention is intended to ensure high rates of conversion with respect to the alkali metal salts of mercaptobenzthiazoles used, associated with high purity and high yields with respect to the benzothiazolyl sulfenamides.

Surprisingly, it was found that, under the conditions according to the present invention, benzothiazolyl sulfenamides are obtained which, as mentioned, are produced with high purity and high storage stability and with high yields associated with high rates of conversion with respect to the alkali metal mercaptobenzthiazole used.

### SUMMARY OF THE INVENTION

Thus, the present invention provides a process for preparing benzothiazolyl sulfenamides by reacting primary amines with alkali metal salts of mercaptobenzthiazole in the presence of hydrogen peroxide and alkali metal hypochlorites, characterized in that an aqueous solution of a metal salt of mercaptobenzthiazole is reacted with primary amines in the presence of at most 90 mol. % of hydrogen peroxide, with respect to the alkali metal salt of the mercaptobenzthiazole used, at a pH in the range 10.0 to 11.0, wherein the amount of hydrogen peroxide is added over a period of ≧2 hours, and then, within a period of ≧10 minutes, alkali metal hypochlorite is added to the reaction mixture for further reaction of the alkali metal salt of the mercaptobenz-thiazole used and the pH of the reaction mixture is increased to 11.5 to 12 after the end of reaction, wherein the amount of primary amine used is 100 to 500 mol. % and the sum of the hydrogen peroxide used and alkali metal hypochlorite used is at most 130 mol. %, with respect to the amount of alkali metal salt of the mercaptobenzthiazole used.

### DETAILED DESCRIPTION OF THE INVENTION

Primary amines which are suitable for use include cyclohexylamine, tert.-butylamine, tert.-amylamine and ispropylamine, cyclohexylamine and tert.-butylamine being used, for example. Suitable alkali metal salts of mercaptobenzthiazole include sodium and potassium salt, sodium salt, for example.

The alkali metal salts of mercaptobenzthiazole can be used in the aqueous form in the process according to the present invention. The concentration of the aqueous solution of alkali metal salts of mercaptobenzthiazole is normally 10 to 60%, of, for example, 20 to 50%.

The process according to the present invention can be preferably performed in the presence of 50 to 90 mol. % of hydrogen peroxide, for example, 70 to 90 mol. % of hydrogen peroxide, with respect to the amount of alkali metal salt of the mercaptobenzthiazole used.

The amount of hydrogen peroxide used depends, inter alia, on the reaction conditions and on the type of metal salt of mercaptobenzthiazole used and also on the primary amine used. The most beneficial amount can be determined each time by means of appropriate preliminary trials.

For the process according to the present invention, it is important that the amount of hydrogen peroxide is added over a certain period. For example, the amount of hydrogen peroxide is added to the reaction mixture within a period of 2 to 5 hours, or, further for example, within a period of 3 to 4 hours. The duration of the addition of hydrogen peroxide again depends on the reaction conditions and on the starting products selected, and can easily be determined by means of appropriate preliminary trials.

The process according to the present invention can be performed at a pH in the range 10.2 to 10.8.

After the intended amount of hydrogen peroxide has been added to the reaction mixture, alkali metal hypochlorite is then added to the reaction mixture within a period of, for example, 15 to 90 minutes, or, further for example, 20 to 60 minutes, for further reaction of the alkali metal salt of mercaptobenzthiazole used. Sodium hypochlorite and potassium hypochlorite, for example, sodium hypochlorite, are used as alkali metal hypochlorites.

After the end of reaction of the alkali metal salts of mercaptobenzthiazole used, the pH of the reaction mixture is raised, for example, to 11.6 to 11.8. Increasing the pH after the end of reaction is important to obtain storage-stable end products.

The amount of primary amine used is, for example, 133 to 400 mol. %, with respect to the amount of alkali metal salt of the mercaptobenzthiazole used. Here again, the most beneficial amount of primary amine to be used can be determined each time by means of appropriate preliminary trials.

In the process according to the present invention, the amount of alkali metal hypochlorite used is chosen so that the sum of the hydrogen peroxide used and of alkali metal hypochlorite used, for example, amounts to 115 mol. %. If, for example, an amount of hydrogen peroxide of 80 mol. % is chosen, then the amount of alkali metal hypochlorite used is at most 35 mol. %.

The concentration of the aqueous hydrogen peroxide solution used is about 5 to 50%, for example, 5 to 35%, further, for example, 5 to 15%, the concentration of the aqueous solution of alkali metal hypochlorite used is about 12 to 15, for example, 13 to 14%.

The process according to the present invention may be performed either continuously or batchwise, wherein the batchwise procedure is preferred.

The process according to the present invention is performed at temperatures of about 35 to 60° C., for example, 40 to 55° C.

After the end of reaction, the benzothiazolyl sulfenamides obtained are isolated, for example by filtration, and the product obtained is washed and dried.

As mentioned, the salts of mercaptobenzthiazole can be used in technical grade quality in the process according to the present invention, i.e. with purity greater than 93%, or, for example, with a purity of 93 to 95%. It is also possible to use mercaptobenzthiazoles with a higher purity, but this may have a detrimental effect on the economic viability of the process according to the present invention.

By using the process according to the present invention, the benzothiazolyl sulfenamides are obtained with purities >99% and in yields of >92% of theoretical. The rates of conversion with respect to the alkali metal salts of the mercaptobenz-thiazole used are >99%.

Furthermore, it may be advantageous that only very small amounts of chloride ions are formed during the process according to the present invention, which means that the process is environmentally friendly when compared with a process which uses only chlorine or only sodium hypochlorite as oxidizing agent. Although, in the process according to the present invention, oxidation is performed mainly with hydrogen peroxide and only small amounts of sodium hypochlorite are used, it is regarded as surprising that the benzothiazolyl sulfenamides are obtained in the high qualities described, with exceptionally high yields and high rates of conversion, starting from the alkali metal salts of mercaptobenzthiazole.

### EXAMPLES

### Example 1

995.94 g of an aqueous 20% strength solution of sodium 2-mercaptobenzthiazole were placed in a reactor and 139.35 g of cyclohexylamine (100% strength) were added to this aqueous solution with intensive stirring (mixing power 0.4 Watts/l). The purity of the sodium mercaptobenzthiazole used was 95%. The molar ratio of pure sodium mercaptobenzthiazole to cyclohexylamine was 1:1.4 mol; i.e. 133 mol. % of cyclohexylamine were used to 100 mol. % of sodium mercaptobenzthiazole with a purity of 95%. The reactor was fitted with a reflux condenser, a thermometer and a pH measurement electrode. The pH of the reaction mixture was adjusted to 10.6 at 40° C. with the aid of 20% strength sulfuric acid and the temperature was kept at 40° C. during the entire reaction.

0.821 moles of hydrogen peroxide (10% strength) were added uniformly to the reaction mixture over a period of 5 hours. This means that 78 mol. % of hydrogen peroxide were used to 100 mol. % of 95% strength sodium mercaptobenzthiazole.

After adding the hydrogen peroxide, 202 g of 13% strength sodium hypochlorite were added to the reaction mixture over the course of 38 minutes and, after the end of reaction, the pH was increased to 11.8 by adding 50% strength aqueous sodium hydroxide. The reaction mixture was then cooled to 30° C., filtered, washed with 10% strength cyclohexylamine, then post-washed again with water and the precipitated product was dried. 245.56 g of benzothiazolyl sulfenamide were obtained, which corresponds to a yield of 97.76% with respect to pure sodium mercaptobenzthiazole. The rate of conversion was

99.4%, with respect to pure NaMBT. The product obtained had a purity of 99.2%, determined by titration as described in ASTM D 4936 (MBT).

During the reaction, the sum of the hydrogen peroxide used and the alkali metal hypochlorite used amounted to a total of 112 mol. % with respect to the amount of 95% strength sodium mercaptobenzthiazole used.

### Example 2

The same procedure as described under example 1 was used, but with the difference that 1.06 moles of 10% hydrogen peroxide (101 mol. % with respect to 100. mol. % strength sodium mercaptobenzthiazole) were used. The hydrogen peroxide was added over the course of 4 hours at a reaction temperature of 40° C.

The mixture was oxidized further over the course of 10 minutes with 57.8 g of a 13% strength NaOCl solution.

The yield was 96.8%, the purity was 99.2% and the rate of conversion was 99.4%.

### Example 3

The same procedure as described under example 2 was used, but the hydrogen peroxide was added over the course of 3 hours at a reaction temperature of 40° C. The mixture was oxidized further over the course of 12 minutes with 61.6 g of a 13% strength NaOCl solution.

The yield was 96.6%, the purity was 98.1% and the rate of conversion was 99.4%.

### Example 4

This example was performed in the same way as example 1, but using 1.10 moles of cyclohexylamine and 90 mol. % of hydrogen peroxide (10% strength). The hydrogen peroxide was added to the reaction mixture over the course of 4 hours at a reaction temperature of 45° C. The mixture was oxidized further over the course of 35 minutes with 182 g of a 13% strength NaOCl solution.

The yield was 95.2%, the purity was 97.5% and the rate of conversion was 99.4%.

### Example 5

The reaction was performed in the same way as in example 1. Sodium mercaptobenzthiazole with a purity of 93% was used. The amount of cyclohexylamine was 1.43 mol, with respect to the 100% pure sodium mercaptobenzthiazole used. 90 mol. % of 10% strength hydrogen peroxide were added to the reaction mixture over the course of 4 hours at a reaction temperature of 40° C. The mixture was oxidized further over the course of 24 minutes with 12 g of a 13% strength NaOCl solution.

The yield was 97.8%, the purity was 98.9% and the rate of conversion was 99.4%.

### Comparison Example 6

397.89 g of an aqueous 50% strength solution of sodium 2-mercaptobenzthiazole were placed in a reactor and 187.57 g of 100% strength cyclohexylamine and 1648 g of water were added to this aqueous solution with mixing. The purity of the sodium mercaptobenzthiazole used was 95%. Then 368.4 g of a 20% strength sulfuric acid were added to the reaction mixture at 50° C.

1 mole of hydrogen peroxide (30% strength) was added uniformly to the reaction mixture at 50° C., over a period of 2 hours. After the end of reaction, 73.68 g of an aqueous 50% strength solution of sodium hydroxide were added to the reaction mixture.

The reaction mixture was then post-stirred for 30 minutes cooled to 30° C., filtered, washed with water and the precipitated product was dried.

222.1 g of benzthiazole sulfenamide were obtained, which indicates a yield of 84%, with respect to pure sodium mercaptobenzthiazole.

The product obtained had a purity of 99%, determined by titration as described in ASTM D 4936.

The mother liquor still contained 34.29 g of sodium mercaptobenzthiazole, which makes the rate of conversion 81.87%.

### Comparison Example 7

175 g of 2-mercaptobenzthiazole, 500 g of water and 118 g of 100% cyclohexylamine (1.2 mol) were placed in a reactor with intensive mixing. The purity of the mercaptobenzthiazole used was 95.5%.

The molar ratio of pure mercaptobenzthiazole to cyclohexylamine was 1:1.2 mol. The fine suspension of the cyclohexylamine salt of 2-mercaptobenzthiazole was heated to 50° C.

1.1 moles of hydrogen peroxide (12.6%) were added uniformly to the reaction mixture over the course of 5 hours, with intensive mixing.

The reaction mixture was then post-stirred for 30 min, cooled to room temperature, filtered, washed with 10% strength cyclohexylamine, then washed again with water and the precipitated product was dried. 250.2 g of benzothiazole sulfenamide were obtained.

The mother liquor contained 4.3 g of non-reacted 2-mercaptobenzthiazole.

The rate of conversion was 97.4% and the yield of cyclohexylsulfenamide was 94.8%.

The product obtained had a purity of 99.1% determined by titration as described in ASTM D 4936.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process for preparing benzothiazolyl sulfenamides comprising:
reacting an aqueous solution of a metal salt of mercaptobenzthiazole with a primary amine(s) in the presence of ≦90 mol. % of hydrogen peroxide, with respect to the alkali metal salt of the mercaptobenzthiazole used, at a pH in the range 10.0 to 11.0,
wherein the amount of hydrogen peroxide is added over a period of ≧2 hours,
adding, within a period of ≧10 minutes, an alkali metal hypochlorite increasing the pH of the reaction mixture to 11.5 to 12,
wherein the amount of primary amine used is 100 to 500 mol. % and the sum of the hydrogen peroxide used and alkali metal hypochlorite used is ≦130 mol. %, with respect to the amount of alkali metal salt of the mercaptobenzthiazole.

2. A process according to claim 1, wherein the primary amine(s) is chosen from the group consisting of cyclohexylamine, tert.-butylamine, tert.-amylamine and ispropylamine and mixtures thereof.

3. A process according to claim 1, wherein the alkali metal salts of the mercaptobenzthiazole are used in a 10 to 60% strength aqueous solution.

4. A process according to claim 1, wherein the amount of hydrogen peroxide is added to the reaction mixture within a period of 2 to 5 hours.

5. A process according to claim, wherein the pH is increased to range 11.6 to 11.8.

6. A process according to claim 1, wherein the process is performed batchwise.

7. A process according to claim 1, wherein the salts of mercaptobenzthiazole is in the range 93 to 95%.

8. A process according to claim 1, wherein the alkali metal hypochlorite is chosen from the group consisting of sodium hypochlorite, potassium hypochlorite and mixtures thereof.
